(19) United States
(12) Patent Application Publication
(10) Pub. No.: US 2022/0000468 A1
(43) Pub. Date: Jan. 6, 2022

(54) SOFT TISSUE ANCHOR

(71) Applicants: Kevin BONNER, Virginia Beach, VA (US); SMITH & NEPHEW, INC, Memphis, TN (US); SMITH & NEPHEW ORTHOPAEDICS AG, Baa (CH); SNITH & NEPHEW ASIA PACIFIC PTE. LIMITED, SIGAPORE (SG)

(72) Inventors: Kevin Bonner, Virgina Beach, VA (US); Kangqiao Li, Mansfield, MA (US); Jennifer Ng, Andover, MA (US)

(21) Appl. No.: 17/279,268

(22) PCT Filed: Mar. 26, 2019
(86) PCT No.: PCT/US19/23969

§ 371 Date: Mar. 24, 2021

Related U.S. Application Data
(60) Provisional application No. 62/648,488, filed on Mar. 27, 2018.

(51) Int. Cl. A61B 17/04; A61F 2/08

(52) U.S. Cl. CPC A61B 17/0401; A61F 2/0811; A61B 2017/0414; A61F 2002/0852; A61B 2017/0412; A61F 2002/0888; A61B 2017/0409

(57) ABSTRACT

Embodiments of the invention include an anchoring system (1) and method of inserting an anchor (100). Suture (305) and graft (550) may be implanted with anchors (100) of some embodiments. Some anchor embodiments may include at least one side that is cut away (115) to provide a protected space, such as central portion (116), for suture (305) and graft (550) and may provide a connection mechanism, such as a connector hole (107), that may be pushed or pulled by an inserter (200) of the anchoring system (1).

### CROSS-REFERENCE TO RELATED APPLICATIONS

This application claims priority to U.S. Prov. Appl. No. 62/648,488, filed on Mar. 27, 2018, all of the disclosure of which is incorporated herein by reference in its entirety.

### FIELD OF THE INVENTION

The present invention relates generally to the field of surgical anchors, and more particularly relates to an improved anchor system and method of inserting an anchor configured to protect one or both of tissue and suture attached to the anchor during use. Some embodiments may include an inserter that is selectively detachable from the anchor.

### BACKGROUND

It is conventional to use tissue anchors to secure sutures, grafts, or other components in tissue such as bone. For example, a blind hole or other tunnel in bone may be created in which one or more of a suture, graft, or other component is to be coupled. A common, conventional technique is to insert an “interference screw” into a blind hole or bone tunnel with a suture, graft, or other component. It is often difficult to insert the interference screw or other conventional implant into the bone tunnel with the suture, graft, or other component without lacerating, crushing, twisting, or otherwise harming the suture, graft, or other component. Such a technique is particularly difficult where the anchor is a threaded anchor, which is often the case with prior art devices.

It would be advantageous to provide an anchoring system capable of securing a suture, synthetic graft structure, soft tissue graft, or other component in a blind hole or other bone tunnel without imposing potentially damaging friction or stress on the suture, synthetic graft structure, soft tissue graft, or other component. Devices of improved design may provide a “tap-in” or linearly insertable anchor that protects suture, soft tissue graft, synthetic graft structures, or other components during insertion. It may additionally be advantageous to provide teeth or other protrusions of increased size near a proximal end of an anchor to facilitate engagement of the anchor near cortical bone. Some systems may also provide an inserter with multiple components that may be retrieved with a single proximal motion of a component of the inserter.

### SUMMARY

An embodiment of the invention is an anchoring system with an anchor and an inserter. The anchor may include an anchor body with a distal end and a proximal end and sides between the distal end and the proximal end, a cutout in a first side of the anchor body such that an interior, central portion of the anchor body near the distal end is accessible from the proximal end along a straight line between the proximal end and the distal end, one or more anti-backout features along one or more of the sides of the anchor body configured to resist proximal movement of the anchor when the anchor has been inserted in a hole with a diameter equal to or less than a maximum diameter of the anchor, and a first inserter connection mechanism. The inserter may include a handle, and a shaft movably coupled to the handle wherein the shaft includes a second inserter connection mechanism configured to engage with the first inserter connection mechanism of the anchor. The shaft may also include a stop along a portion of the shaft's length that is engageable with the handle to restrict movement between the shaft and the handle to enable the handle to be moved proximally with the shaft upon engagement of the stop with the handle when the shaft is being moved proximally.

Another embodiment of the invention is an anchor including at least an anchor body with a distal end and a proximal end and sides between the distal end and the proximal end and a cutout in a first side of the anchor body such that an interior, central portion of the anchor body near the distal end is accessible from the proximal end along a straight line between the proximal end and the distal end. The anchor may also include one or more anti-backout features along one or more of the sides of the anchor body configured to resist proximal movement of the anchor when the anchor has been inserted in a hole with a diameter equal to or less than a maximum diameter of the anchor and a first inserter connection mechanism at the interior, central portion of the anchor body. The first inserter connection mechanism may be configured to provide a connection by which the anchor may be pulled proximally.

An additional embodiment of the invention is an inserter that includes at least a handle and a shaft movably coupled to the handle wherein the shaft includes an inserter connection mechanism configured to engage with an anchor. The shaft may include a stop along a portion of the shaft's length that is engageable with the handle to restrict movement between the shaft and the handle to enable the handle to be moved proximally with the shaft upon engagement of the stop with the handle when the shaft is moved proximally.

Still another embodiment of the invention is a method of inserting an anchor into a bone that includes at least coupling a shaft portion of an inserter that passes through a handle of the inserter into the anchor by placing a second connection mechanism on the shaft of the inserter into a first connection mechanism of the anchor; inserting the anchor into the bone by moving the inserter handle distally toward the bone; decoupling the second connection mechanism of the inserter from the first connection mechanism of the anchor; moving the shaft of the inserter proximally away from the anchor to engage a stop along a portion of the shaft with the handle; and continuing to move the shaft of the inserter proximally away from the anchor to move the handle away from the anchor.

### BRIEF DESCRIPTION OF THE DRAWINGS

FIG. 1 is a perspective view of an embodiment of an anchoring system.

FIG. 2 is a perspective view of an inserter embodiment of the anchoring system of FIG. 1.

FIG. 3 is a perspective view of an anchor coupled with a shaft of the inserter embodiment of the anchoring system of FIG. 1 with the handle removed for clarity.

FIG. 4 is a perspective view of an anchor embodiment of the anchoring system of FIG. 1.

FIG. 5A is a side elevation view of the anchor of FIG. 4.

FIG. 5B is another side elevation view of the anchor of FIG. 4.

FIG. 5C is a distal end elevation view of the anchor of FIG. 4.

FIG. 6 is a side elevation view of a distal end of the anchoring system of FIG. 1.

FIG. 7A is a side elevation view of the distal end of FIG. 6 with the anchor removed from the anchoring system.

FIG. 7B is a side elevation view with a partial cross-sectional view of the distal end of FIG. 6 with the anchor removed from the anchoring system.

FIG. 8A is a side elevation view of the distal end of FIG. 6 with the anchor removed from the anchoring system and a shaft moved proximally relative to a handle.

FIG. 8B is a side elevation view with a partial cross-sectional view of the distal end of FIG. 6 with the anchor removed from the anchoring system and the shaft moved proximally relative to the handle.

FIG. 9 is a perspective view of the anchoring system of FIG. 1 indicating rotation of the shaft to couple with the anchor and movement of the shaft distally toward a hole in a bone.

FIG. 10 is a perspective view of a distal portion of the anchoring system of FIG. 1 with suture and graft coupled to the anchor.

FIG. 11 is a perspective view of the distal portion of the anchoring system illustrated in FIG. 10 with the suture advanced further toward the anchor and a knot tied in strands of the suture.

FIG. 12 is a perspective view of the distal portion of the anchoring system illustrated in FIG. 11 being advanced into a hole in tissue such as bone.

FIG. 13 is a perspective view of the distal portion of the anchoring system illustrated in FIG. 11 further advanced into the hole.

FIG. 14 is a is a perspective view of the anchoring system of FIG. 1 with suture and graft coupled to the anchor and further indicating actions needed to detach the shaft of the inserter from the anchor and remove the inserter from the anchor.

FIG. 15 is a perspective view of the graft shown in FIG. 14 protruding from a hole in tissue such as bone with the anchor inserted in the hole and the inserter removed.

### DETAILED DESCRIPTION

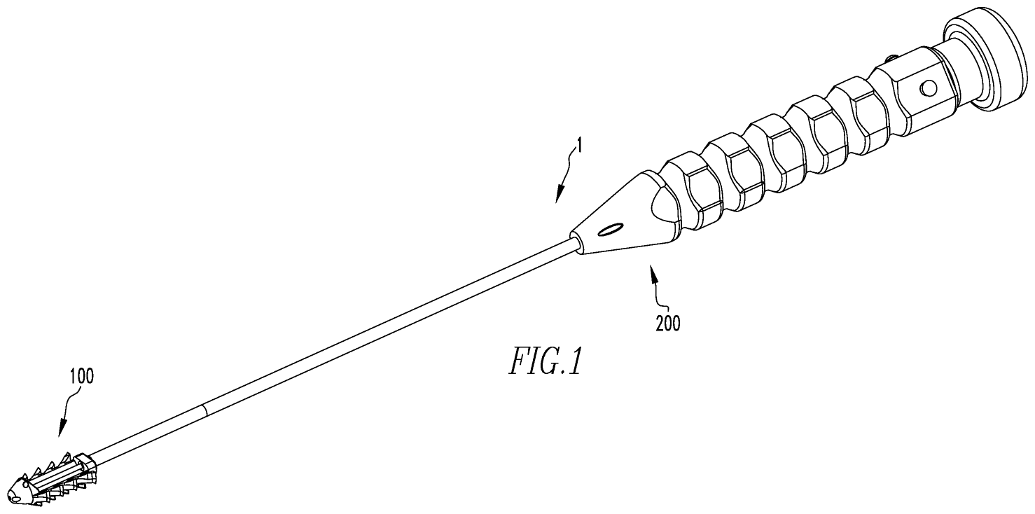

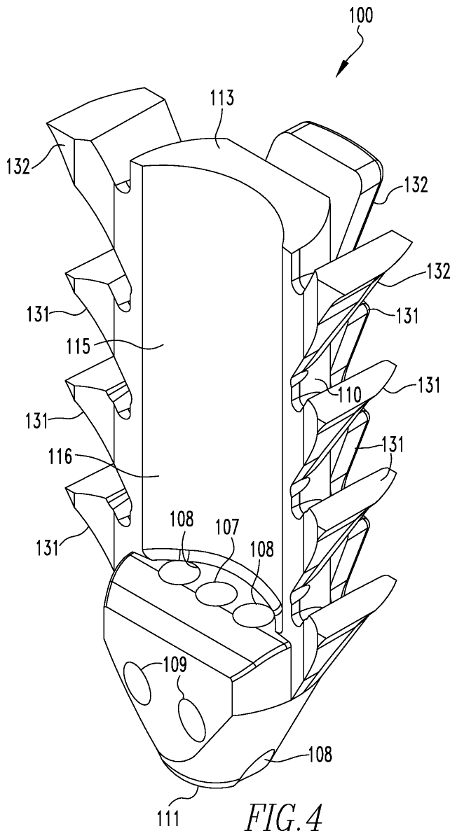

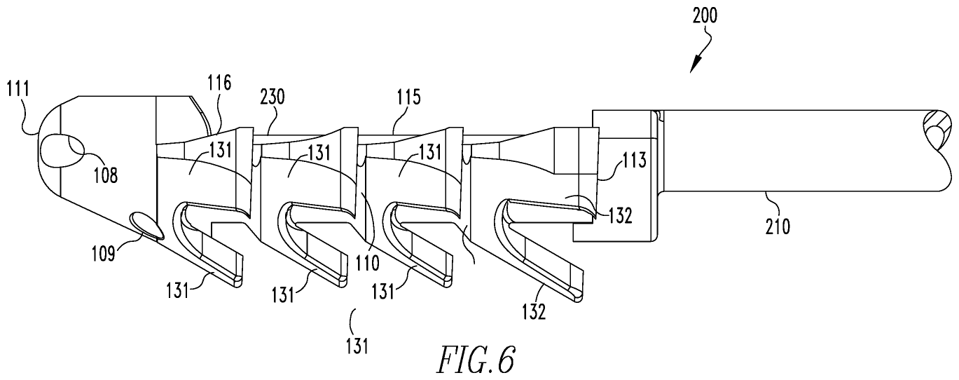

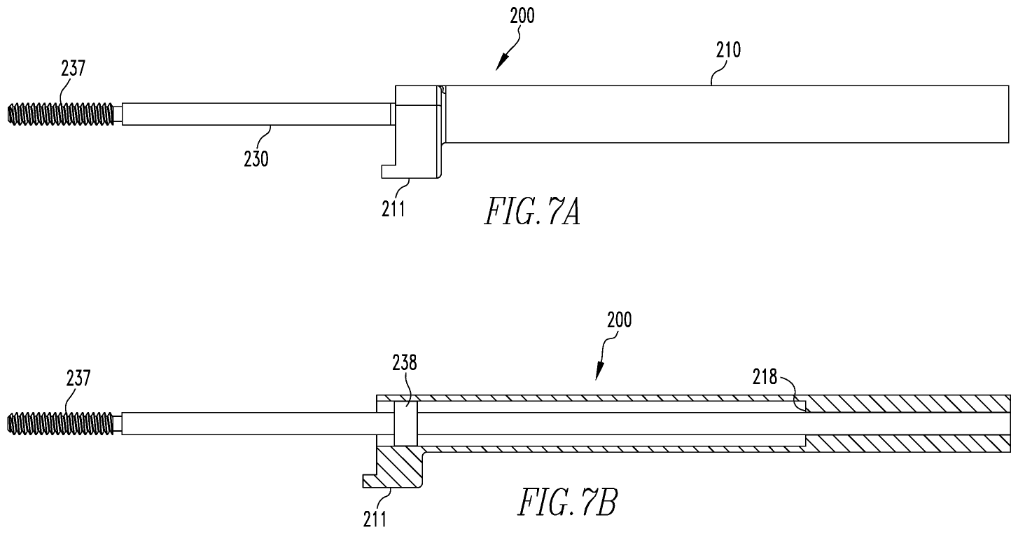

An embodiment of an anchoring system 1 and its component parts and methods of implementation are illustrated in FIGS. 1-15. The anchoring system 1 may include an anchor 100 and an inserter 200. Various features of the anchor 100 are illustrated in combination in FIGS. 1, 3-6, and 9-12. The anchor 100 shown in more detail in FIGS. 4-6 includes an anchor body 110 with a distal end 111, a proximal end 113, and sides along a length between the distal end 111 and the proximal end 113. The illustrated anchor 100 includes a cutout 115 in a first side of the anchor body 110. The anchor 100 with cutout 115 provide for an interior, central portion 116 of the anchor body 110 near the distal end 111 that is accessible from the proximal end 113 along a straight line between the proximal end 113 and the distal end 111. In the illustrated embodiment, an entire volume along the straight line between the proximal end 113 and the distal end 111 is not enclosed along the first side from the interior, central portion 116 to the proximal end 113 of the anchor body 110. In other embodiments, a full or partial enclosure may be employed between an interior central portion and a proximal end.

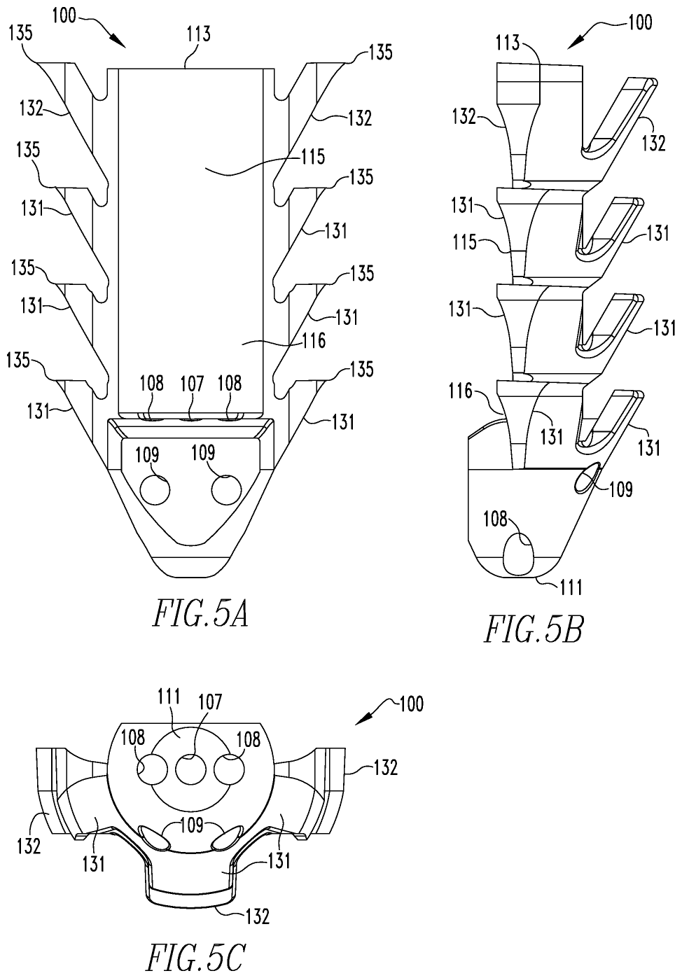

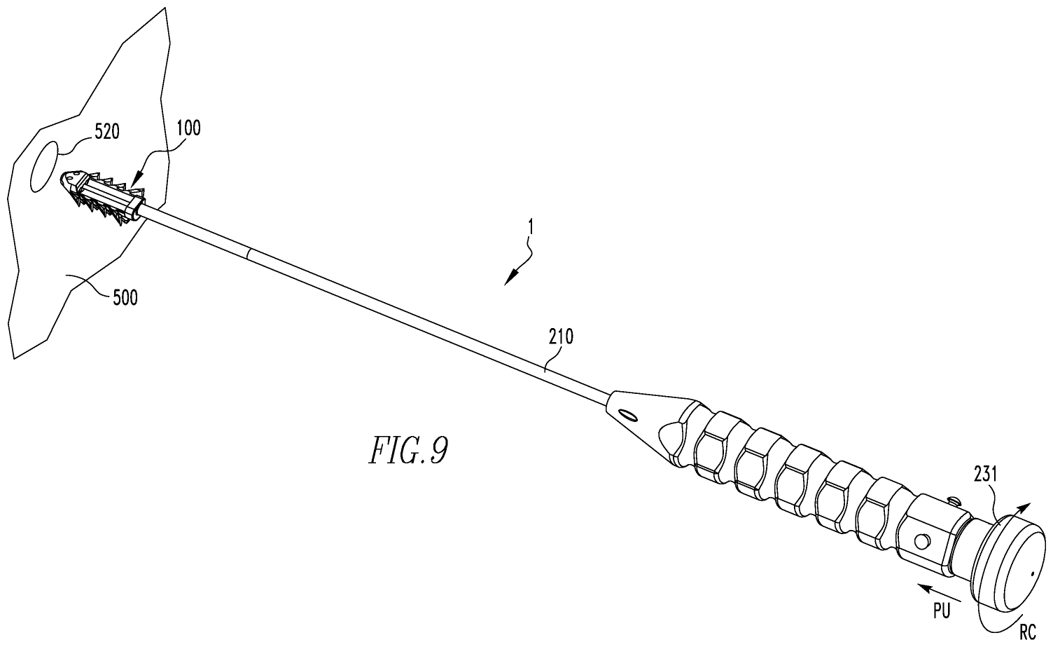

The illustrated embodiment also shows anti-backout features in the form of teeth (standard teeth 131, extended teeth 132) along the length of the anchor body 110 on three sides, and no teeth on the first side of the anchor body 110. Other embodiments may include teeth on more or fewer sides on an anchor body, as may be effective. The teeth 131, 132 shown are configured to resist proximal movement of the anchor 100 when the anchor 100 has been inserted in a hole 520 (FIGS. 9 and 12-15) in a bone 500 with a diameter equal to or less than a maximum diameter of the anchor 100. As used in association with the term maximum diameter herein, the term “diameter” is a straight-line distance through an anchor from one side of the anchor to the other side of the anchor that passes through a cross-sectional central portion of the anchor. A “diameter” as used herein is not limited to an object with a circular or even rounded cross-section. As used herein, a hole with a diameter equal to or less than a maximum diameter of the anchor 100 is not considered to be an exact specification, particularly in light of the fact that holes in bone may be relatively imprecise or irregular. The teeth 131, 132 depicted may be resilient or flexible such that the teeth 131, 132 flex away from the wall of the hole 520 (FIGS. 9 and 12-15) when the anchor 100 is inserted into the hole 520. A resilient or flexible tooth may also include an angular edge, such as the angular edge 135 (FIG. 5A), that catches against the wall of the hole 520 if a proximal force is applied to the anchor 100 while in the hole 520. Anti-backout features of these or other embodiments may include barbs, hooks, spikes, or any other effective mechanism to resist backout of an anchor to which the features are attached. For example and without limitation, anti-backout elements may be triangular, square, pyramid shaped, shark's tooth shaped, and raked to any effective degree. In the disclosed embodiment, the extended teeth 132 near the proximal end 113 extend further from a longitudinal central axis of the anchor body 110 than one or more of the standard teeth 131 nearer the distal end 111 of the anchor body 110. This configuration of the extended teeth 132 in some embodiments provides for stronger fixation under cortical bone at or near where the extended teeth 132 may engage with bone.

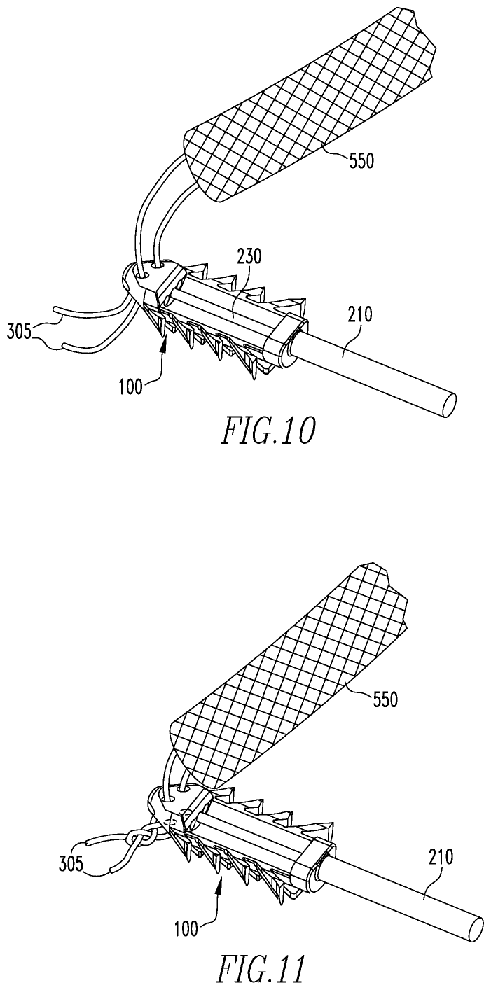

The anchor 100 includes a first inserter connection mechanism in the form of an opening in the interior, central portion 116 of the anchor body 110, referred to in association with this embodiment as a connector hole 107. The connector hole 107 is a threaded hole into which a portion of the inserter 200 may be advanced to couple the anchor 100 to the inserter 200. An opening or connector hole of any other effective shape, with or without threads, may be used in other embodiments. The anchor 100 includes longitudinal holes 108 (FIGS. 4-6) and transverse holes 109 (FIGS. 4-6) which may be used as part of an inserter connection mechanism or may be configured to receive suture, such as the suture 305 (FIGS. 10 and 11). The illustrated longitudinal holes 108 and transverse holes 109 are distal of the interior, central portion 116 of the anchor body 110. In some embodiments, one or more stay sutures may be passed through holes 108, 109 to enable removable coupling between the anchor 100 and inserter 200. Any other effective connecting mechanism may be used to removably couple between an inserter and an anchor of this or other embodiments. For example and without limitation, an anchor of some embodiments may be held to an inserter by friction or by some mechanical feature such as a snap, hook, or ball and detent. In the illustrated embodiment, the first inserter connection mechanism is configured to provide a connection by which the anchor may be pulled proximally when selectively engaged.

The inserter 200 is illustrated in whole and by its component parts in FIGS. 1-3 and 6-14. The inserter 200 depicted includes a handle 210 and a shaft 230 movably coupled to the handle 210. The handle 210 shown includes a seat 211 at its distal end that is configured to couple with the proximal end 113 of the anchor 100 and limit rotation of the anchor 100 while coupled in the seat 211. The handle 210 shown includes a shoulder 218 that is a narrower opening through the handle 210 capable of limiting relative movement between the handle 210 and the shaft 230. The shaft 230 is movable relative to the handle 210 by rotation about its longitudinal axis and to a limited degree back and forth longitudinally relative to the handle 210.

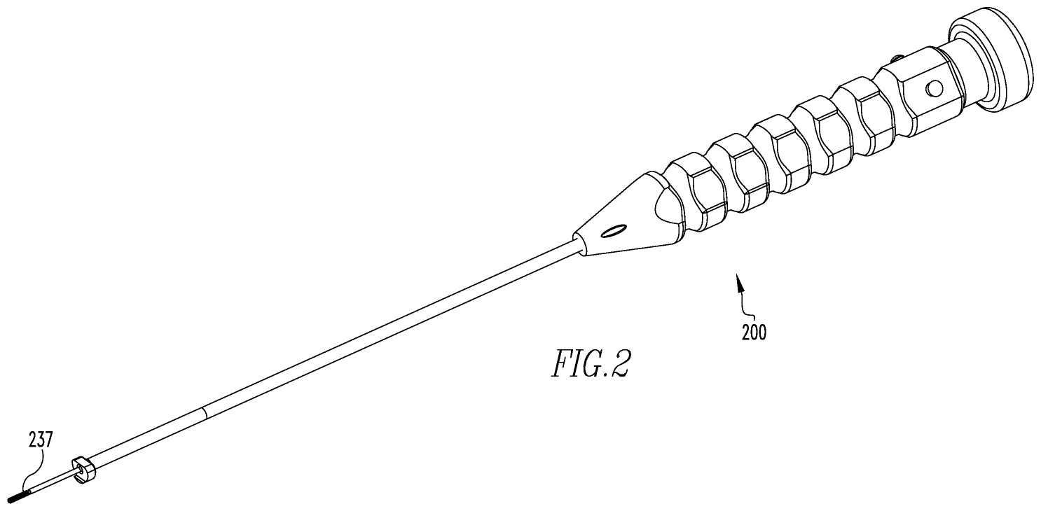

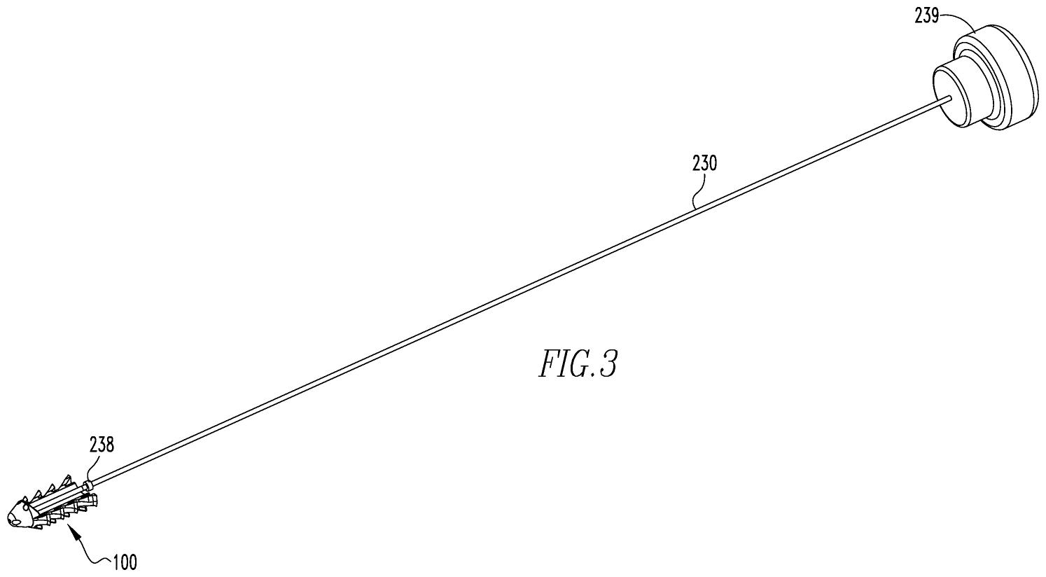

The shaft 230 illustrated includes a second inserter connection mechanism, in the form of threads 237 (FIGS. 2 and 7A-8B), configured to engage and disengage with the first inserter connection mechanism (the connector hole 107) to provide a releasable coupling. The shaft 230 depicted also includes a stop 238 (FIGS. 3, 7B, and 8B) along a portion of the shaft's length that is engageable with the shoulder 218 of the handle 210 to restrict movement between the shaft 230 and the handle 210 to enable the handle 210 to be moved proximally with the shaft 230 upon engagement of the stop 238 with the handle 210 at the shoulder 218 when the shaft is being moved proximally. The illustrated stop 238 is a widened volume of the shaft 230 that does not fit through at least a portion of the handle 210 through which other portions of the shaft 230 are configured to fit. In other embodiments, a stop may be of any effective configuration to limit movement between a shaft and a handle.

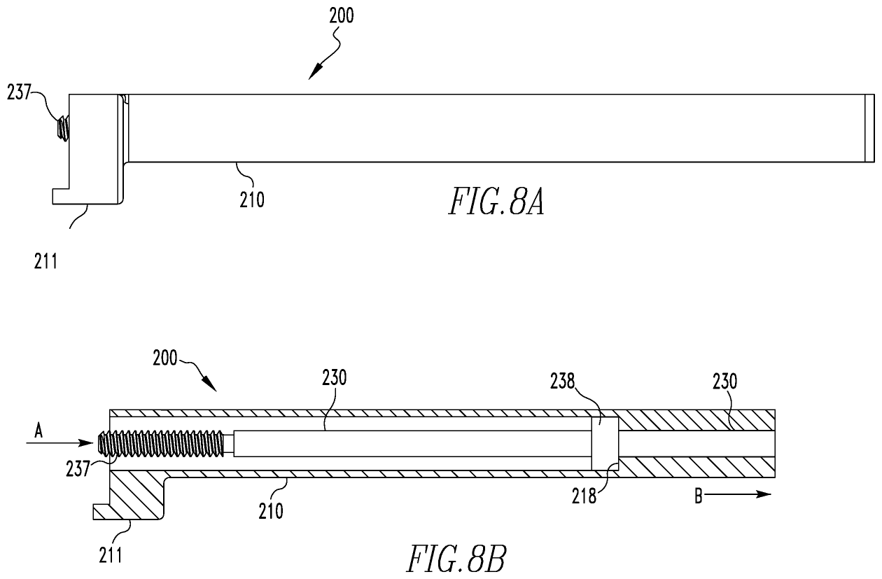

The shaft 230 of the inserter 200 in the illustrated embodiment also includes a knob 239 at a proximal end of the shaft 230. The knob 239 may be attached to the shaft 230 during the inserter assembly process. For example, a proximal end of the shaft 230 may be inserted through the distal end of the handle 210. Subsequently, the knob 239 may be coupled to the proximal end of the shaft 230 such that the shaft 230 is able to slide proximally within the handle 210 until the stop 238 contacts the shoulder 218, as illustrated in FIG. 8B; and the shaft 230 is able to slide distally within the handle 210 until the knob 239 contacts the proximal end of the handle, as illustrated in FIGS. 1, 2, and 10. The knob 239 depicted is smooth and round in cross-section, but other embodiments of a knob may include tactile gripping surfaces and may be any other effective cross-sectional shape.

A synthetic, woven graft 550 is illustrated in FIGS. 10-15. The graft of this or other embodiments may be any effective synthetic material, or a tendon, ligament, muscle, cartilage, or other tissue component. As illustrated in FIGS. 10 and 11, sutures 305 have been whipstitched to the synthetic, woven graft 550. The sutures 305 illustrated include two strands of suture that have been threaded through transverse holes 109. In FIG. 10, the sutures 305 have been advanced through respective transverse holes 109, but have not been advanced to a final position or tied to one another. In FIG. 11, the sutures 305 are shown further advanced into the transverse holes 109 and are tied in a knot with one another. In other embodiments, graft tissue, a patient's tissue, or sutures of any type, or a combination of such elements may be passed through an opening such as one or both of the transverse holes 109 or the longitudinal holes 108 for anchoring. Sutures of the anchoring system may be any type of suture, for example and without limitation, a monofilament, multistrand, or woven construct. A suture may be passed through an anchor while the anchor is outside of a patient's body, or may be passed when the anchor is in whole or in part within a joint or other subcutaneous portion of a patient's body. Any other effective structure, which may or may not include use of a suture, may be used to enable coupling of a soft tissue graft to an anchor in other embodiments.

Another embodiment of the invention is a method of inserting an anchor, such as the anchor 100, into a bone, such as the bone 500. As an example, the method may include coupling a shaft portion, such as the threads 237, of an inserter that passes through a handle, such as the handle 210 into an anchor, such as the anchor 100 depicted. In the illustrated embodiment, the threads 237 that are the second connection mechanism on the shaft 230 are placed into the connector hole 107, which is the first connection mechanism of the anchor 100. As illustrated in FIG. 9, the shaft 230 may be turned by rotating the knob 231 clockwise, as indicated by arrow RC, which turns the threads 237 in the connector hole 107 to couple the second connection mechanism on the shaft 230 with the first connection mechanism of the anchor 100. Other embodiments may include turning, sliding, expanding, or any other effective mechanism to couple first and second connection mechanisms. As illustrated in FIGS. 10 and 11, methods may also include connecting a graft material to the anchor 100 with sutures, as described above in more detail. In other embodiments, other effective techniques may be used to couple a graft or suture material to an anchor to accomplish a desired surgical task.

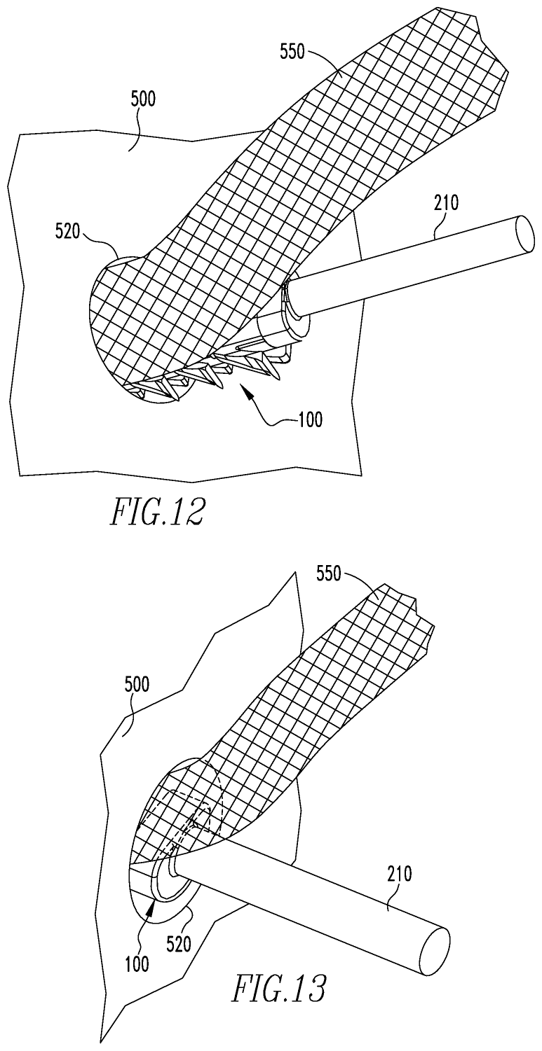

The method may also include inserting the anchor 100 into the bone 500 by moving the inserter handle 210 distally toward the bone 500. For example, the handle 210 may be pushed linearly into the bone 500 as reflected by the arrow PU in FIG. 9, with further progress of the insertion illustrated in FIGS. 12 and 13. In other embodiments, inserting an anchor into the bone may also include turning the anchor about its longitudinal axis while pushing the anchor toward the bone, such as with a threaded anchor.

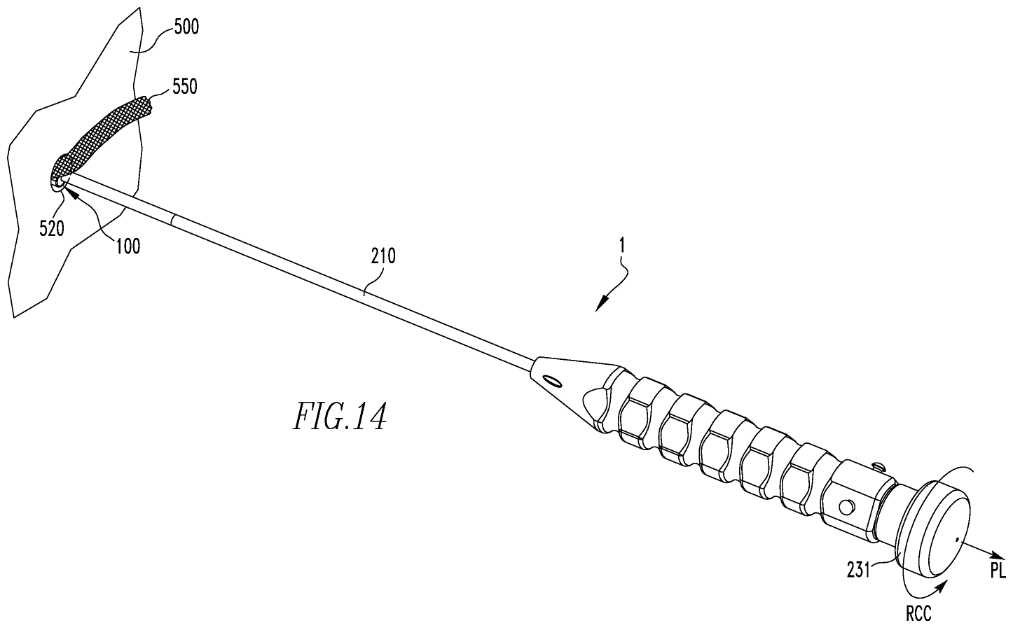

The method may additionally include decoupling the second connection mechanism of the inserter from the first connection mechanism of the anchor. In the illustrated embodiment, this is accomplished as shown in FIG. 14 by rotating the knob 231 counterclockwise (arrow RCC), which in turn rotates the threads 237 out of the connector hole 107, which decouples the second connection mechanism of the inserter 200 from the first connection mechanism of the anchor 100.

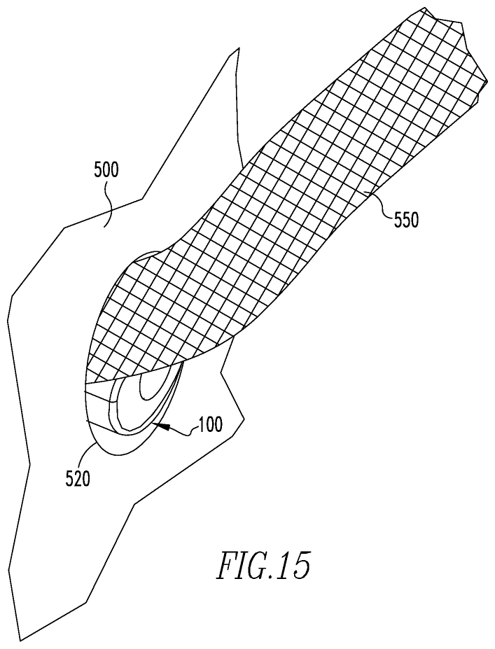

The method may also include moving the decoupled shaft 230 of the inserter 200 proximally away from the anchor 100 to engage a stop 238 along a portion of the shaft 230 with the handle 210. In the illustrated embodiment, the engagement with the handle 210 is at the shoulder 218, as shown in FIG. 8B. Moving of the shaft 230 away from the anchor 100 is illustrated by arrow PL in FIG. 14, and also reflected by arrow A in FIG. 8B. A reaction of the engage handle 210 to the proximally moved knob 237 and shaft 230 is shown by the arrow B in FIG. 8B. The shaft 230 may continue to be moved proximally away from the anchor 100 to move the handle proximally away, eventually leaving the anchor 100 and the graft 550 in place in the hole 520 in the bone 500, as illustrated in FIG. 15.

Various embodiments of a system wholly or its components individually may be made from any biocompatible material. For example and without limitation, materials may include in whole or in part: non-reinforced polymers, reinforced polymers, metals, ceramics, adhesives, reinforced adhesives, and combinations of these materials. Reinforcing of polymers may be accomplished with carbon, metal, or glass or any other effective material. Examples of biocompatible polymer materials include polyamide base resins, polyethylene, Ultra High Molecular Weight (UHMW) polyethylene, low density polyethylene, polymethylmethacrylate (PMMA), polyetheretherketone (PEEK), polyetherketoneketone (PEKK), a polymeric hydroxyethylmethacrylate (PHEMA), and polyurethane, any of which may be reinforced. Example biocompatible metals include stainless steel and other steel alloys, cobalt chrome alloys, zirconium, oxidized zirconium, tantalum, titanium, titanium alloys, titanium-nickel alloys such as Nitinol and other superelastic or shape-memory metal alloys. The material of some embodiments of the anchor or its components may include a resorbable material, which over time is incorporated into a patient's tissue in which the device is implanted. Effective resorbable materials of some embodiments provide an adequate level of strength and stiffness for a time frame that exceeds that expected for tissue healing. After healing, such devices may be completely or otherwise significantly absorbed by a patient's body. The biological performance for some embodiments, particularly for application at a bony site, may be enhanced by incorporation of bioactive fillers in the polymer. A non-limiting list of synthetic and natural biodegradable polymers includes polyglycolide, polyhydroxobutyrate, chitosan, hyaluronic acid, and hydrogels. Poly(2-hydroxyethyl-methacrylate) and poly(ethylene glycol) may be used, as may poly(L-lactide) (PLA). REGENESORB is a trade named resorbable polymer material that may be used and includes D,L-polylactid acid. Embodiments of the anchor may be made via a molding process or other process known to one of skill in the art. The internal and external surfaces of embodiments of the distal anchor component may be formed during a molding process or may be formed by drilling or machining. Sutures or other similar components of the invention may be single strand, woven, braided, or any combination thereof from any of these or other biocompatible materials. The sutures or other similar components may be any effective natural or synthetic material and may be a use or combination of materials well-known in the art. Sutures or other similar components of various embodiments may be resorbable or not resorbable.

Terms such as proximal, distal, along, near, under, and the like have been used relatively herein. However, such terms are not limited to specific coordinate orientations, distances, or sizes, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. An anchoring system comprising:
an anchor comprising:
an anchor body with a distal end and a proximal end and sides between the distal end and the proximal end,
a cutout in a first side of the anchor body such that an interior, central portion of the anchor body near the distal end is accessible from the proximal end along a straight line between the proximal end and the distal end,
one or more anti-backout features along one or more of the sides of the anchor body configured to resist proximal movement of the anchor when the anchor has been inserted in a hole with a diameter equal to or less than a maximum diameter of the anchor, and
a first inserter connection mechanism; and
an inserter comprising:
a handle, and
a shaft movably coupled to the handle,
wherein the shaft includes a second inserter connection mechanism configured to engage with the first inserter connection mechanism of the anchor, and
wherein the shaft includes a stop along a portion of the shaft's length that is engageable with the handle to restrict movement between the shaft and the handle to enable the handle to be moved proximally with the shaft upon engagement of the stop with the handle when the shaft is being moved proximally.

2. The anchoring system of claim 1 wherein a volume along the straight line is not enclosed by the first side from the interior, central portion of the anchor body to the proximal end of the anchor body.

3. The anchoring system of claim 1 wherein the one or more anti-backout features includes a tooth.

4. The anchoring system of claim 3 wherein the tooth is resilient and is configured to be flexed away from a wall of the hole when the anchor is inserted into the hole, and wherein the flexible tooth includes an angular edge that catches against the wall of the hole if a proximal force is applied to the anchor.

5. The anchoring system of claim 1 wherein one or more of the anti-backout features near the proximal end extend further from a longitudinal central axis of the anchor body than one or more anti-backout features nearer the distal end.

6. The anchoring system of claim 1 wherein there are no anti-backout features on the first side of the anchor body.

7. The anchoring system of claim 1 wherein the first inserter connection mechanism is an opening in the interior, central portion of the anchor body.

8. The anchoring system of claim 7 wherein the opening in the interior, central portion of the anchor body is a threaded hole.

9. The anchoring system of claim 1 wherein the anchor body includes at least one distal hole located distal of the interior, central portion of the anchor body through which a suture may be threaded.

10. The anchoring system of claim 1 wherein the second inserter connection mechanism is configured to releasably couple with the first connection mechanism.

11. The anchoring system of claim 1 wherein the second inserter connection mechanism includes a threaded portion configured to couple with the first connection mechanism.

12. The anchoring system of claim 1 wherein the stop is a widened volume of the shaft that does not fit through at least a portion of the handle through which other portions of the shaft are configured to fit.

13. An anchor comprising:
an anchor body with a distal end and a proximal end and sides between the distal end and the proximal end;
a cutout in a first side of the anchor body such that an interior, central portion of the anchor body near the distal end is accessible from the proximal end along a straight line between the proximal end and the distal end;
one or more anti-backout features along one or more of the sides of the anchor body configured to resist proximal movement of the anchor when the anchor has been inserted in a hole with a diameter equal to or less than a maximum diameter of the anchor; and
a first inserter connection mechanism at the interior, central portion of the anchor body;
wherein the first inserter connection mechanism is configured to provide a connection by which the anchor may be pulled proximally.

14. A method of inserting an anchor into a bone comprising:
coupling a shaft portion of an inserter that passes through a handle of the inserter into the anchor by placing a second connection mechanism on the shaft of the inserter into a first connection mechanism of the anchor;
inserting the anchor into the bone by moving the inserter handle distally toward the bone;
decoupling the second connection mechanism of the inserter from the first connection mechanism of the anchor;
moving the shaft of the inserter proximally away from the anchor to engage a stop along a portion of the shaft with the handle; and
continuing to move the shaft of the inserter proximally away from the anchor to move the handle away from the anchor.

15. The method of claim 14 wherein the act of placing a second connection mechanism on the shaft of the inserter into a first connection mechanism of the anchor includes turning the shaft of the inserter to engage the second connection mechanism into the first connection mechanism.

16. The method of claim 15 wherein the act of turning the shaft of the inserter to engage the second connection mechanism into the first connection mechanism includes engaging threads on the shaft into a threaded hole of the anchor.

17. The method of claim 14 wherein the act of inserting the anchor into the bone by moving the inserter handle distally toward the bone includes pushing the anchor linearly into the bone.

18. The method of claim 14 wherein the act of inserting the anchor into the bone by moving the inserter handle distally toward the bone includes turning the anchor about its longitudinal axis while pushing the anchor toward the bone.

19. The method of claim 14 wherein the act of moving the shaft of the inserter proximally away from the anchor to engage a stop along a portion of the shaft with the handle includes engaging a stop that is a widened volume of the shaft that does not fit through at least a portion of the handle through which other portions of the shaft are configured to fit with the handle.

20. The method of claim 14, further comprising passing a suture coupled to a graft through at least one distal hole located distal of an interior, central portion of the anchor body and placing the graft in the interior central portion of the anchor body.

21. The method of claim 20 wherein the graft is placed in the interior central portion of the anchor body prior to the anchor being inserted into the bone.
